(19) United States
(12) Patent Application Publication
(10) Pub. No.: US 2009/0005263 A1
(43) Pub. Date: Jan. 1, 2009

(54) Signal Amplification of Biorecognition Events Using Photopolymerization in the Presence of Air

(76) Inventor: Laura Rae Kuck, Boulder, CO (US)

Correspondence Address: HOLLAND & HART, LLP, P.O BOX 8749, DENVER, CO, 80201, US

(21) Appl. No.: 12/279,523

(22) PCT Filed: Feb. 9, 2007
(86) PCT No.: PCT/US2007/061914

§ 371 Date: Aug. 14, 2008

Related U.S. Application Data
(60) Provisional application No. 60/773,532, filed on Feb. 15, 2006.

(51) Int. Cl. C40B 30/04; C12Q 1/68

(52) U.S. Cl. 506/9; 435/6

(57) ABSTRACT

The present invention discloses an inexpensive and non-enzymatic signal amplification technique on both DNA and protein microarrays. The technique is uses photo-initiated polymerization and is conducted directly on the microarray. A capture molecule is bound to the desired surface. The target molecule then binds to the capture molecule. A label sequence with a bound photo initiator binds to the target molecule. Polymerization is activated using a wave length of light corresponding to the wave length needed to activate the chosen photo initiator. This new non-enzymatic method can be applied to the rapid detection of any biological pathogen via either microarray or ELISA platforms. Influenza is described herein as an example application of the technology.

### CROSS REFERENCE APPLICATIONS

This application is claims priority from U.S. provisional application No. 60/773,532 filed Feb. 15, 2006.

### BACKGROUND

Effective global monitoring of any biological pathogen will require an inexpensive, reliable and simple analytical system that can be widely manufactured and distributed. DNA microarrays, or biochips, represent promising technology for accurate and relatively rapid pathogen identification (Wang et al., 2002). For example, there are currently under development both DNA and protein microarrays for strain analysis of influenza (see below). However, several practical issues currently prevent widespread use of biochips as diagnostic tools, including the lack of rapid and simple processes for extraction of genetic material or antigenic proteins from complex samples, expensive reagents (e.g., fluorescent labels), and expensive and non-field-portable biochip readers/scanners (Schena, 2003).

Influenza is an orthomyxovirus with three genera, types A, B, and C. The types are distinguished by the nucleoprotein antigenicity (Dimmock et al., 2001). Types A and B are the most clinically significant, causing mild to severe respiratory illness. Influenza B is a human virus and does not appear to be present in an animal reservoir. Type A viruses exist in both human and animal populations, with significant avian and swine reservoirs. Influenza A and B each contain 8 segments of negative sense single stranded RNA. Type A viruses can also be divided into antigenic sub-types on the basis of two viral surface glycoproteins, hemagglutinin (HA) and neuraminidase (NA). There are currently 15 identified HA sub-types (designated H1 through H15) and 9 NA sub-types (N1 through N9), all of which can be found in wild aquatic birds (Lamb & Krug, 1996). Of the 135 possible combinations of HA and NA, only four (H1N1, H1N2, H2N2, and H3N2) have widely circulated in the human population since the virus was first isolated in 1933. The two most common sub-types of influenza A currently circulating in the human population are H3N2 and H1N1.

New type A strains emerge due to genetic drift that results in slight changes in the antigenic sites on the surface of the virus. Thus, the human population experiences epidemics of “the flu” each year. However, more drastic genetic changes can result in an antigenic shift (a change in the subtype of HA and/or NA) resulting in a new subtype capable of rapid spread in a susceptible population. The influenza A virus of 1918 was of the H1N1 subtype and it replaced the previous virus (probably H3N8 as deduced by seroarcheology) that had been the dominant type A virus in the human population (Hilleman, 2002). Antigenic shift most likely arises from genetic reassortment when two different sub-types infect the same cell (Webster et al. 1992). Since the viral genetic information is stored in eight separate segments, packaging of new virions within a cell that is replicating two different viruses (e.g. an avian type A and a human type A) can result in a virus with a mixture of genes from each of the parent viruses. This mechanism is presumed to be the means by which avian-like surface glycoproteins (and some internal, nonglycoprotein genes) appeared in the viruses responsible for the 1957 (H2N2) and 1968 (H3N2) pandemics. This reassortment of surface antigens is an ongoing possibility as shown by the recent appearance of H1N2 reassortants worldwide (Xu et al. 2002).

The gold standard for complete antigenic characterization of influenza remains viral isolation in either egg or tissue culture (Brammer et al., 2002) followed by a hemagglutination inhibition (HAI) analysis of cross-reactivity as described in the WHO manual on influenza diagnosis and surveillance (Webster et al., 2002). In this test, several reference antisera (typically ˜20) are used to evaluate how well an unknown virus binds to standard antibodies grown against well-characterized viruses. The new isolated virus is then categorized as most “like” an antigenically related known virus. The isolation/HAI testing process is relatively expensive, tremendously time consuming (days), labor intensive, and non-quantitative. While rapid and relatively inexpensive tests for diagnosis of influenza A and B are commercially available (Harper et al., 2005), none provide the detailed strain analysis required for useful surveillance and vaccine formulation. For genetic characterization, the CDC and other WHO Influenza Collaborating Centers now routinely employ reverse-transcriptase polymerase chain reaction (RT-PCR) followed by sequencing. However, at this point in time genetic information alone is insufficient to describe the antigenic properties of the influenza virus and is used as complimentary information to antigenic characterization.

With the advent of rapid genome sequencing and large genome databases, it is now possible to utilize genetic information in a myriad of ways. One of the most promising technologies is DNA microarrays (Vernet, 2002; Heller, 2002), also commonly referred to as DNA chips or biochips. DNA chip technology has found widespread use in gene expression analysis and there are now several demonstrations of biochips used in diagnostics (Vernet, 2002). Anthony et al. recently demonstrated rapid identification of 10 different bacteria in blood cultures using a BioChip (Anthony et al., 2000). The microarray assay was conducted in about 4 hrs. The approach utilized universal primers for PCR amplification of the variable region of bacterial 23s ribosomal DNA and a 3×10 array of 30 unique capture sequences. This work demonstrates one of the most exciting aspects of biochip platforms—the capability to screen for multiple pathogens simultaneously. DeRisi and co-workers demonstrated a “virus chip” that contained sequences for hundreds of viruses, including many that cause respiratory illness (Wang et al., 2002). This chip proved useful in identifying the corona virus associated with SARS. In the DeRisi work PCR technology was used to amplify the genetic material for capture, and expensive fluorescent labels were used to generate signals from positive spots. Antibody microarrays are also becoming increasingly attractive as a platform for direct detection of pathogens, with the understanding that accuracy, reliability, cost and total assay time will have to be improved to match or surpass the current generation of single-test diagnostic kits (Taussig and Landegren, 2003; Ward et al. 2004).

The Rowlen group at the University of Colorado is currently developing both genetic and antigenic microarrays (FluChip) for rapid strain analysis of influenza. The overall objective of the research is to provide investigators with a new and powerful tool for rapid strain analysis and improved surveillance of influenza. While microarray-based sub-typing of influenza has been demonstrated (Kessler et al., 2004; Sengupta et al., 2003; Li et al., 2001), the objective of the FluChip project is to develop a tool for complete and rapid strain analysis. The basic approach for the genetic FluChip is shown in FIG. 1. A target or capture sequence 101 is attached to the FluChip 100. A number of different capture sequences can be utilized. The RNA target sequence 102 will bind to the capture sequence, which then subsequently binds the label sequence 103. Currently, it is necessary to amplify the viral RNA using reverse transcription, PCR, and transcription. For field portable applications it is desirable to reduce the assay complexity and it is essential that detection be achieved inexpensively.

While a FluChip based on genetic information is expected to be of great utility for strain analysis, due to the high mutation rate in influenza it may not provide a complete picture. For example, the CDC has noted that significant genetic changes do not necessarily result in significant antigenic changes. Conversely, in some cases a single point mutation can result in a distinct antigenic change (Smith, 2005). Therefore, in addition to the genetic FluChip, research is also being conducted to test hypothesis that an antibody array can be developed to provide a rapid antigenic characterization of the influenza virus. The basic concept is shown in FIG. 2. Antibodies 201 raised against a wide range of influenza hemagglutinin and neuraminidase proteins are spatially arranged in a microarray format. After treatment of the patient sample in much the same manner as that used in the current rapid flu test (e.g., Biostar's FLU OIA), the proteins 202, or whole virus, is captured and subsequently labeled with a secondary fluor-tagged antibody 203. Of course, the limitation of such a chip is the number of antibodies available and the potential for missing an influenza virus that has antigenically shifted. However, it is important to note that the antigenic microarray would serve in the same capacity as the current predominant method for antigenic characterization—the hemagglutination inhibition test (i.e., it would provide a measure of how well the new virus binds to standard antibodies).

There would be significant advantage to enabling field-portable and inexpensive detection and imaging of microarrays. However, even with the best and most expensive scanners, which are not field portable, the limit of detection in clinical samples is a significant issue. In recent years, several methods for detecting a small number of oligo hybridization events on a surface have been proposed and demonstrated. Examples include the branched DNA assay, developed by the Chiron Corporation (Emeryville, Calif.), rolling circle amplification (Nallur et al., 2001) and dendrimer technology (Stears et al., 2000). Although the methods mentioned above are reliable and sensitive, they are not ideal for “on-site” surveillance due to expense and difficulty of use. All of them rely on fluorescence detection and do not enable the use of an inexpensive and field portable microarray reader.

The University of Colorado filed for patent protection (PCT/US2004/029733 published as WO200/024386) of the photopolymerization signal amplification (PSA) concept in 2004. The disclosed invention used a hydroxyethyl acrylate monomer and a custom made “macrophotoinitiator”, in which multiple photoinitiators were present on a single molecule. The macrophotoinitiator was composed of a water-soluble copolymer of acrylic acid and acrylamide to which a commercial water-soluble photoinitiator (Ciba I2959) and Neutravidin were covalently attached using standard coupling chemistry (EDC/NHS). In this case, the label sequence was biotinylated and the macrophotoinitiator bound to the target by the strong binding between biotin and avidin. The advantages of this approach include a single label for all oligos (biotin), which can be applied directly to the target oligo using photobiotin (McInnes et al., 1990), thereby reducing the number of oligos required, and the high local concentration of photoinitiators (˜150 photoinitiators per chain) from relatively few binding events (1-2 active Neutravidins per chain). Using this system, the prior art method was able to demonstrate visual detection (i.e., the polymer thickness was sufficient to enable detection by eye) of as few as 1000 biotinylated oligos on a Biostar OIA substrate (Covalciuc et al., 1999), which represented ˜2 orders of magnitude improvement over the Biostar OIA limit of detection (based on enzymatic signal amplification). The significant disadvantages of the prior art approach include the use of a toxic, non-water soluble and volatile monomer, use of ultraviolet light for initiation (365 nm), and most importantly the necessity of purging all reagents and the mixture with argon or nitrogen in order to remove oxygen due to its inhibitory effects on photopolymerization reaction chemistry.

### SUMMARY OF THE INVENTION

The present invention uses photo-initiated polymerization to detect a desired biorecognition event and is conducted directly on the microarray or other desired surface. The most significant advantages of the invention described herein include the use of a visible light photoinitiator, a water soluble non-toxic monomer, and reaction chemistry that allows photopolymerization in the presence of air. In this invention a probe molecule is bound to the desired surface. The target molecule is bound to the photoinitiating label in solution and this complex is bound to the probe molecule. Polymerization is activated using a wave length of light corresponding to the wave length needed to activate the chosen photo initiator. This new non-enzymatic method can be applied to the rapid detection of any biological pathogen via either microarray or ELISA platforms. Influenza typing and subtyping is described herein as an example application of the technology.

In an embodiment, the invention provides a method for amplifying a molecular recognition interaction between a target and a probe comprising the steps of:

a) contacting the target with a photoinitiator label under conditions effective to form a target-photoinitiator label complex;

b) contacting the target-label complex with the probe under conditions effective to attach the target-photoinitiator label complex to the probe;

c) substantially removing any unbound target-photoinitiator label complex;

d) contacting the photoinitiator label-target-probe complex with a polymerizing solution comprising a polymer precursor and a photoinitiator in the presence of air;

e) exposing the photoinitiator label-target-probe complex and the polymerizing solution to visible light in the presence of air, thereby forming a polymer; and

f) detecting the polymer formed, thereby detecting an amplified target-probe interaction.

In another embodiment, the invention provides methods for identification of a target species based on its molecular interaction with an array of different probe species, each probe species being attached to a solid substrate at known locations.

In the methods of the invention, if the target species undergoes a molecular recognition reaction with a probe, the probe will be labeled with a polymer. Detection of the polymer-labeled probes allows identification of which probes have undergone the molecular recognition reaction and therefore identification of the target.

In an embodiment, the invention provides a method for identifying a target comprising the steps of

a) providing a probe array comprising a plurality of different probes, wherein the probes are attached to a solid substrate at known locations;

a) contacting the target with a photoinitiator label under conditions effective to form a target-photoinitiator-label complex;

b) contacting the target-photoinitiator label complex with the probe under conditions effective to attach the complex to the probe;

c) substantially removing any unbound target-photoinitiator label complex;

d) contacting the photoinitiator label-target-probe complex with a polymerizing solution comprising a polymer precursor and a photoinitiator in the presence of air;

e) exposing the photoinitiator-label-target-probe complex and the polymerizing solution to visible light in the presence of air, thereby forming a polymer; and

f) detecting the polymer formed, wherein the polymer location indicates the probe which forms a target-probe complex with the target, thereby identifying the target.

These and other features and advantages of the disclosed method in the chosen components and the combination thereof, the mode of operation and use, as will become more apparent from the following description, reference being made to the accompanying drawings that form a part of this specification wherein like reference characters designate corresponding parts in the several views. The embodiments and features thereof are described and illustrated in conjunction with systems, tools and methods which are meant to exemplify and to illustrate, not being limiting in scope.

### BRIEF DESCRIPTION OF THE DRAWINGS

FIG. 1 is a schematic diagram of basic microarray design.

FIG. 2 is a schematic diagram of capture and label approach for an antibody array.

FIG. 3 is a schematic diagram of hybridization and photoinitiation process.

FIG. 4a is a schematic diagram of the antibody array layout, where the letter/number designations represent the antibody against a specific hemagglutinin protein.

FIG. 4b is a representative image of the stained polymer after capture of hemagglutinin (8 ng) and photopolymerization.

FIG. 5 is a fluorescence signal-to-background as measured in stained polymer produced from captured hemagglutinin.

FIG. 6 contains fluorescence and transmission images of a influenza microarray array after photopolymerization.

FIG. 7 is a calibration of eosin-labeled, amine-terminated oligo spotted onto aldehyde glass.

Before explaining the disclosed embodiments in detail, it is to be understood that the embodiments are not limited in application to the details of the particular arrangements shown, since other embodiments are possible. Also, the terminology used herein is for the purpose of description and not of limitation.

### DETAILED DESCRIPTION OF THE INVENTION

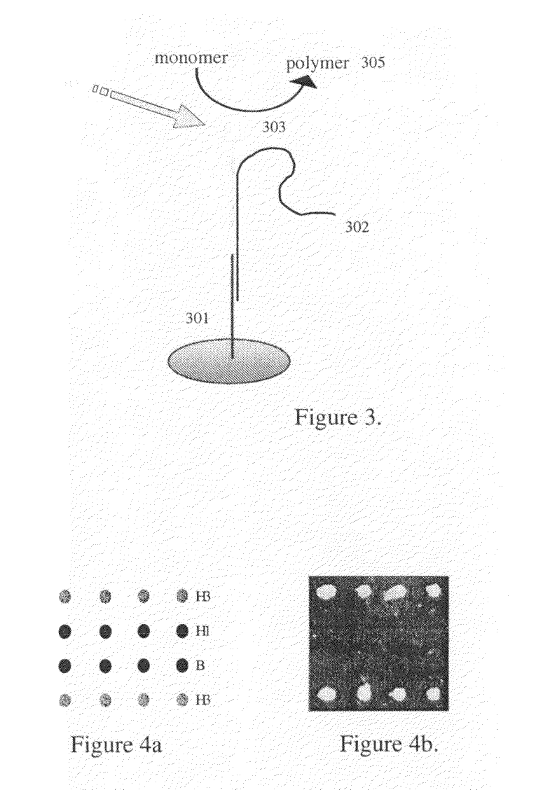

The basic concept for an innovative approach to signal amplification via photopolymerization is shown in FIG. 3. Rather than a fluor-labeled-oligo, a photo-active label 303 is used to label the target sequence 302 which is then bound to the probe (capture) sequence 301. The monomer 304 reagent solution is added after hybridization. Light of the appropriate wavelength is used to initiate formation of a solid polymer 305. Polymer forms only where the photoinitiator was bound to the microarray.

The idea is based on a photoactive molecular label, termed a photoinitiator, rather than a fluor label. Once labeled, the system is covered in a solution that contains “monomers” 304 and other facilitating reagents and irradiated with an appropriate wavelength of light. The light creates free radicals, which propagate by radical addition to and between the surrounding monomers 304. Under the correct conditions, the result is a solid polymer 305. If the monomer 304 were fluorescent, the solid could be “read” using standard fluorescence detection. Other options include the use of a chromophoric monomer or staining the solid with a fluorophore or chromophore after polymer formation. The tremendous advantages of this system include enormous signal amplification without the use of an enzyme, formation of a solid (which can preserve the sample), applicability to both nucleic acids and proteins, low reagent cost, and the potential for visual or inexpensive detection.

It is desirous to have a non-toxic and non-volatile system that would function well in an oxygen environment (i.e., ambient conditions) with visible light excitation (in order to avoid the potential unwanted photochemistry caused by UV light). In addition, elimination of the need for a macrophotoinitiator is desirable.

The present invention utilizes a non-toxic, non-volatile, and water soluble monomer such as poly(ethyleneglycol diacrylate) (PEGDA 575, Sigma Aldrich) as the monomer, eosin isothiocyanate (EITC, Sigma Aldrich) as the amine-reactive photoinitiator and two additional reagents, 1-vinyl-2-pyrrolidinone (Sigma Aldrich) and triethanolamine (Sigma Aldrich), that ensure reaction in the presence of air. In a custom reaction, EITC is covalently attached to selected amine-terminated oligos and monoclonal antibodies. As this reaction is obvious to one skilled in the art, it will not be further discussed. Light at 532 nm is used to initiate the reaction. In order to minimize bulk photopolymerization the initiating light is optimally used in a pulsed or fluctuating matter.

All of the following examples were demonstrated with the mixture described above. However, alternate photoinitiators, co-initiators, monomers, and reagents may also be used. Some possible alternates include:
Amine Co-Initiator. There are a number of amines that have been demonstrated to be effective “co-initiators” or “co-catalysts” in photopolymerization reactions such as: triethylamine, ethanolamine, N-methyl diethanolamine, N,N-dimethyl benzylamine, dibenzyl amine, N-benzyl ethanolamine, N-isopropyl benzylamine, tetramethyl ethylenediamine, tetramethyl ethylenediamine, lysine, ornithine, histidine and arginine.
Monomers. PEGDA is the ideal monomer due to its water solubility, low toxicity, and low propensity for surface contamination. However, a range of PEGDA lengths could be used. PEGDA is commercially available in monomer lengths of 200, 575, and 700 from Sigma Aldrich. In addition, the VP could be replaced with ethylene glycol diacrylate or other reactive monomers.
Alternate Photoinitiators. Molecules other than eosin may serve as visible light photoinitiators, such as: methylene blue, rose bengal, congo red, malachite green, merocyanine 540, hypericin and hypocrellin.

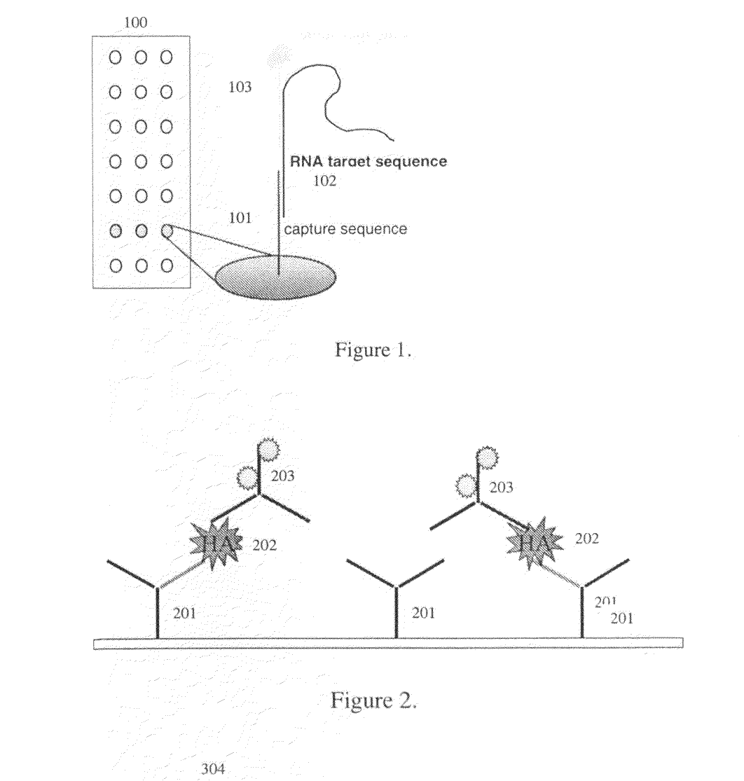

Although FIG. 1 illustrates hybridization of complementary RNA to RNA, the detection and amplification scheme generalizes to many other types of molecular recognition events. Agents capable of participating in molecular recognition events include, but are not limited to, agonists and antagonists for cell membrane receptors, toxins and venoms, viral epitopes, hormones (e.g., opiates, steroids, etc.), hormone receptors, peptides, enzymes, enzyme substrates, substrate analogs, transition state analogs, cofactors, drugs, proteins, and antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. In different embodiments, the detection and amplification scheme can be used to detect and amplify the molecular recognition interaction between nucleic acids, an antibody and an antigen, and a first and a second protein.

Microarrays can be used to detect hybridization as well as protein-protein interactions, protein drug binding, and enzymatic catalysis (Schena, M., “Microarray Analysis, (2003) John Wiley & Sons, New Jersey, p. 153). As used herein, molecular recognition interactions are those in which the probe recognizes and selectively binds a target, resulting in a target-probe complex. Molecular recognition interactions also involve the formation of noncovalent bonds between the two species. The binding occurs between specific regions of atoms (molecular domains) on the probe species which have the characteristic of binding or attaching specifically to unique molecular domains on specific target species. Molecular recognition interactions can also involve responsiveness of one species to another based on the reciprocal fit of a portion of their molecular shapes.

In order for molecular interaction between the target and the probe to identify the target, the molecular interaction between the target and the probe must be sufficiently specific. For hybridization, the selectivity is a measure of the specificity of the molecular recognition event. “Selectivity” or “hybridization selectivity” is the ratio of the amount of hybridization (i.e., number of second nucleic acids hybridized) of fully complementary hybrids to partially complementary hybrids, based on the relative thermodynamic stability of the two complexes. For the purpose of this definition it is presumed that this ratio is reflected as an ensemble average of individual molecular binding events. Selectivity is typically expressed as the ratio of the amount of hybridization of fully complementary hybrids to hybrids having one base pair mismatches in sequence. Selectivity is a function of many variables, including, but not limited to: temperature, ionic strength, pH, immobilization density, nucleic acid length, the chemical nature of the substrate surface and the presence of polyelectrolytes and/or other oligomers immobilized on the substrate or otherwise associated with the immobilised film.

For hybridization, the homology of the target and probe molecules influences whether hybridization occurs. Cross-hybridization can occur if the sequence identity between the target and the probe is greater than or equal to about 70% (Schena, M., “Microarray Analysis, (2003) John Wiley & Sons, New Jersey, p. 151).

In an embodiment, either the target or the probe is a nucleic acid. In an embodiment, both the target and the probe are a single stranded nucleic acid. In an embodiment, the probe is an oligonucleotide, a relatively short chain of single-stranded DNA or RNA. “Nucleic acid” includes DNA and RNA, whether single or double stranded. The term is also intended to include a strand that is a mixture of nucleic acids and nucleic acid analogs and/or nucleotide analogs, or that is made entirely of nucleic acid analogs and/or nucleotide analogs and that may be conjugated to a linker molecule. “Nucleic acid analogue” refers to modified nucleic acids or species unrelated to nucleic acids that are capable of providing selective binding to nucleic acids or other nucleic acid analogues. As used herein, the term “nucleotide analogues” includes nucleic acids where the internucleotide phosphodiester bond of DNA or RNA is modified to enhance bio-stability of the oligomer and “tune” the selectivity/specificity for target molecules (Uhlmann, et al., (1990), Angew. Chem. Int. Ed. Eng., 90: 543; Goodchild, (1990), J. Bioconjugate Chem., 1: 165; Englisch et al., (1991), Angew, Chem. Int. Ed. Eng., 30: 613). Such modifications may include and are not limited to phosphorothioates, phosphorodithioates, phosphotriesters, phosphoramidates or methylphosphonates. The 2′-O-methyl, allyl and 2′-deoxy-2′-fluoro RNA analogs, when incorporated into an oligomer show increased biostability and stabilization of the RNA/DNA duplex (Lesnik et al., (1993), Biochemistry, 32: 7832). As used herein, the term “nucleic acid analogues” also include alpha anomers (.alpha.-DNA), L-DNA (mirror image DNA), 2′-5′ linked RNA, branched DNA/RNA or chimeras of natural DNA or RNA and the above-modified nucleic acids. For the purposes of the present invention, any nucleic acid containing a “nucleotide analogue” shall be considered as a nucleic acid analogue. Backbone replaced nucleic acid analogues can also be adapted to for use as immobilised selective moieties of the present invention. For purposes of the present invention, the peptide nucleic acids (PNAs) (Nielsen et al., (1993), Anti-Cancer Drug Design, 8: 53; Engels et al., (1992), Angew, Chem. Int. Ed. Eng., 31: 1008) and carbamate-bridged morpholino-type oligonucleotide analogs (Burger, D. R., (1993), J. Clinical Immunoassay, 16: 224; Uhlmann, et al., (1993), Methods in Molecular Biology, 20,. “Protocols for Oligonucleotides and Analogs,” ed. Sudhir Agarwal, Humana Press, NJ, U.S.A., pp. 335-389) are also embraced by the term “nucleic acid analogues”. Both exhibit sequence-specific binding to DNA with the resulting duplexes being more thermally stable than the natural DNA/DNA duplex. Other backbone-replaced nucleic acids are well known to those skilled in the art and can also be used in the present invention (See e.g., Uhlmann et al., (1993), Methods in Molecular Biology, 20, “Protocols for Oligonucleotides and Analogs,” ed. Sudhir Agrawal, Humana Press, NJ, U.S.A., pp. 335).

More generally, the probe and/or target can be an oligomer. “Oligomer” refers to a polymer that consists of two or more monomers that are not necessarily identical. Oligomers include, without limitation, nucleic acids (which include nucleic acid analogs as defined above), oligoelectrolytes, hydrocarbon based compounds, dendrimers, nucleic acid analogues, polypeptides, oligopeptides, polyethers, oligoethers any or all of which may be immobilized to a substrate. Oligomers can be immobilized to a substrate surface directly or via a linker molecule.

In an embodiment, the probe is DNA. The DNA may be genomic DNA or cloned DNA. The DNA may be copied or complementary DNA (cDNA), or the target may be messenger RNA (mRNA). The DNA may also be an Expressed Sequence Tag (EST) or a Bacterial Artificial Chromosome (BAC). For use in hybridization microarrays, double-stranded probes are denatured prior to hybridization, effectively resulting in single-stranded probes.

DNA microarrays are known to the art and commercially available. The general structure of a DNA microarray is a well defined array of spots on an optically flat surface, each of which contains a layer of relatively short strands of DNA.

For probes bound to a substrate using aldehyde attachment chemistry, the substrate may be treated with an agent to reduce the remaining aldehydes prior to contacting the probe with the target. One suitable reducing agent is sodium borohydride NaBH₄. Such a treatment can decrease the amount of reaction between the monomer and the aldehyde coating on the glass, thus decreasing the amount of background signal during the detection step.

Prior to contacting the target with the probe, the target may be biotinylated to allow later attachment of at least one initiator via biotin-avidin interaction. In an embodiment, photobiotinylation reagents (Pierce, Quanta Biodesign) can be used to biotin-label the target.

In an embodiment, the target may be contacted with the photoinitiator label prior to contacting the target with the probe, so long as use of a photoinitiator-labeled target does not substantially limit its participation in the desired molecular recognition event. In an embodiment, the invention provides a method for amplifying a molecular recognition interaction between a target and a probe comprising the steps of contacting a photoinitiator-labeled target with a probe under conditions effective to form a photoinitiator-labeled target-probe complex, removing target not complexed with the probe, contacting the photoinitiator-labeled target-probe complex with a polymer precursor, exposing the photoinitiator-labeled target-probe complex and the polymer precursor to light, thereby forming a polymer, and detecting the polymer formed, thereby detecting an amplified target-probe interaction.

The probe is contacted with a solution comprising the target under conditions effective to form a target-probe complex. The conditions effective to form a target-probe complex depend on the target and probe species. For ssDNA or RNA targets binding to ssDNA probes, suitable hybridization conditions have been described in the scientific literature. In an embodiment, this solution also comprises an agent, such as a crowding agent, to limit nonspecific interactions. With reference to nucleic acid interactions, a crowding agent is an agent that interrupts nonspecific adsorption between nucleic acids that are not complementary. Formamide is one such agent to limit nonspecific interactions (Stahl, D. A., and R. Amann. 1991. Development and application of nucleic acid probes, p. 205-248. in E. Stackebrandt and M. Goodfellow (ed.), Nucleicacid techniques in bacterial systematics. John Wiley &amp; Sons Ltd., Chichester, United Kingdom). Nonspecific interactions can also be limited by applying a blocking agent to the microarray prior to contacting the target with the probe. Suitable blocking agents are known to the art and include, but are not limited to BSA, nonfat milk, and sodium borohydride. Detergents such as sodium lauroyl sarcosine or sodium dodecyl sulfate can also be added to aldehyde surface hybridization reactions to reduce background (Schena, M., “Microarray Analysis, (2003) John Wiley &amp; Sons, New Jersey, p. 117). The target solution may also be contacted with the probe at higher temperatures in order to limit nonspecific interactions.

After the target is contacted with the probe, targets which have not formed target-probe complexes are removed. The unbound targets can be removed through rinsing. Water or an aqueous solution may be used for rinsing away unbound targets.

If the initiator is to be attached through biotin-avidin interaction, a blocking agent can be applied to the microarray to limit nonspecific interaction of avidin. Suitable blocking agents are known to the art and include, but are not limited to, BSA and nonfat milk. In an embodiment array is incubated with the blocking agent for approximately 20 minutes at about room temperature.

In an embodiment, the target-probe complex is contacted with a photoinitiator label under conditions effective to attach the photoinitiator label to the target probe complex. In an embodiment, the photoinitiator label comprises avidin or streptavidin and at least one photoinitiator. In an embodiment, a plurality of photoinitiators are attached to the avidin or streptavidin to form a polymer photoinitiator label. In another embodiment, a plurality of photoinitiators and avidin or streptavidin are attached to a polymer. If the target has been biotin-labeled, interaction between the avidin or streptavidin and the biotin can attach the photoinitiator label to the target, and thus to the target-probe complex. Information on avidin-biotin interaction is provided in Wilcheck, M., (a) Bayer, E. A. Eds. (1990) “Avidin-biotin technology” Methods in Enzymology 184.

Photoinitiator molecules can be attached to avidin or streptavidin by modification of avidin or streptavidin lysine residues. For photoinitiators having a carboxylic acid functional group, the carboxylic functional group of the photoinitiator can be coupled to the amine of the lysine residue in the presence of a coupling agent. The result is the formation of a peptide bond between the initiator and the protein.

In another embodiment, a polymer photoinitiator label is formed. Such a polymeric photoinitiator label can be formed from a polymer which can be coupled with both the photoinitiator and avidin or streptavidin. In an embodiment, the polymer comprises carboxylic acid groups and amide groups. In an embodiment, the polymer photoinitiator comprises sufficient photoinitiators so that it may be regarded as a macroinitiator (having many initiators present on a single molecule).

In an embodiment, the use of a macroinitiator can increase the initiator concentration by a factor of between about 10 to about 100.

After contact of the photoinitiator label with the target-probe complex, unattached photoinitiator is removed. Unattached photoinitiator may be removed by rinsing with water or an aqueous solution.

The photoinitiator-labeled target-probe complex is contacted with a solution comprising a polymer precursor and a photoinitiator. As used herein polymer precursor” means a molecule or portion thereof which can be polymerized to form a polymer or copolymer. Polymer precursors include any substance that contains an unsaturated moiety or other functionality that can be used in chain polymerization, or other moiety that may be polymerized in other ways. Such precursors include monomers and oligomers. In an embodiment, the solution further comprises a solvent for the polymer precursor. In an embodiment, the solvent is aqueous.

The solution may further comprise cross-linking agents. A crosslinking agent can stabilize the polymer that is formed and improve the amplification factor (Hacioglu B., Berchtold K. A., Lovell L. G., Nie J., &amp; Bowman C. N).

(2002) Polymerization Kinetics of HEMA/DEGDMA: using Changes in initiation and Chain Transfer Rates to Explore the Effects of Chain-Length-Dependent Termination. Biomaterials 23: 4057-4064). Finally, a small amount of inhibitor can be added to the formulation to limit background polymerization caused by impurities and trace radicals formed by absorption by molecules other than the initiator.

In different embodiments, the polymer precursor is a photopolymerizable monomer capable of forming a fluorescent polymer, a magnetic polymer, a radioactive polymer or an electrically conducting polymer. In an embodiment, the polymer precursor is water soluble. In an embodiment, the polymer precursor is a photopolymerizable fluorescent methacrylate monomer. When the polymer precursor is fluorescent, the fluorophore may absorb the light used in the photopolymerization process. To compensate, the exposure time of the polymer precursor to the light and/or the light intensity can be adjusted.

In another embodiment, the polymer precursor is capable of forming a polymer gel. In an embodiment, the gel is covalently crosslinked and a cross-linking agent is added to the polymer precursor containing solution. In another embodiment, the gel is noncovalently crosslinked. In an embodiment, the polymer gel formed is made detectable by absorption of a fluorescent, magnetic, radioactive, or electrically conducting solution by the gel.

In an embodiment, the polymer gel is a hydrogel. The term “hydrogel” refers to a class of polymeric materials which are extensively swollen in an aqueous medium, but which do not dissolve in water. In general terms, hydrogels are prepared by polymerization of a hydrophilic monomer under conditions where the polymer becomes cross-linked in a three dimensional matrix sufficient to gel the solution. The hydrogel may be natural or synthetic. A wide variety of hydrogel-forming compositions are known to the art.

### EXAMPLES OF THE PRESENT INVENTION

### Example 1

### Signal Amplification on an Antibody Microarray

A simple antibody microarray was used to evaluate the utility of the present invention for signal amplification from a captured protein. The arrangement of monoclonal antibodies against A/H3 hemagglutinin (HA, BioDesign International), A/H1 HA, and B/HA in a proof-of-principle microarray is shown in FIGS. 4a and 4b, where lighter shades of grey indicate expected (4a) and observed (4b) positive signals. The schematic of the antibody array layout is shown in FIG. 4a. The letter/number designations represent the antibody against a specific hemagglutinin protein. The A/H1 HA and B/HA served as negative controls in an experiment designed to capture A/H3 hemagglutinin. Recombinant HA from A/Wyoming/3/2003 was graciously provided by Protein Science Corporation at 80 μg/mL in PBS. The labeling strategy was as shown in FIG. 2. The approximate concentration of the labeled antibody after purification was ˜3 μg/mL. A volume of 100 μL of the antibody label was placed in contact with the array for 20 min at room temperature. Photopolymerization was conducted by adding 60 μL of the reagent mixture to the array, followed by 45 s of irradiation with 532 nm light (˜35 mW/cm²) from a small, hand-held laser.

A typical result for capture of H3 HA (in 100 μL) is given in FIG. 4b. FIG. 4b is a representative image of the stained polymer after capture of hemagglutinin (8 ng) and photopolymerization. The resulting polymer was easily visualized by eye (estimated thickness is ˜30 μm). No false positive and no false negatives were observed.

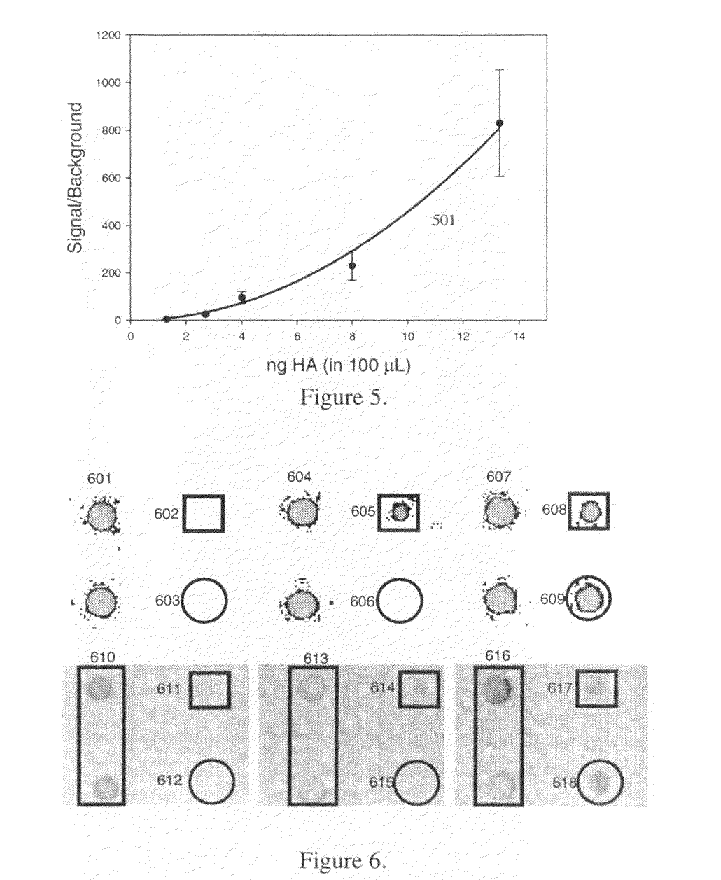

In order to determine the limit of detection of influenza HA using the present invention, a quantitative analysis was performed. The polymer formed after capture and label of the HA contained a small quantity of eosin, which has a broad emission spectrum and a fluorescence quantum yield of ˜0.33 (http://probes.invitrogen.com/). The measured fluorescence (Genetix scanner) signal-to-background (S/B) ratio as a function HA concentration is shown in FIG. 5 for the range of 1.3 to 13 ng of HA. Error in the y-axis represents ±1 standard deviation from 16 measurements. Line 501 is a regression to a second-order function. The measured limit of detection, defined by a S/B˜3, was 1.3 ng of HA in 100 μL (13 ng/mL).

While this number is higher than the LOD often achieved using enzymes, e.g. an HRP-Amplex Red system can achieve ˜0.5 ng/mL detection (Campa, 2004), the present invention eliminates the need for enzymes and has the advantage that the reaction is complete within minutes. Elimination of enzymes is desired due to their sensitivity to environmental conditions and their tendency to degrade during reaction.

These steps were followed to obtain the above results:
1. Standard protocols were used to print the antibody array and capture hemagglutinin.
2. EITC-labeled antibody preparation: 20 μL antibody (3.8 mg/mL), 60 μL bicarbonate buffer and 40 μL EITC in DMF (2 μg/μL) were combined. The mixture was shaken in dark for 90 minutes, then purified using a NAP5 column, eluting with 10 mM PBS. Final volume=500 uL.
3. Labeling: The labeled antibody was diluted 1/50 in 10 mM PBS and 100 μL was added to the captured hemagglutinin using a rubber well. The slide was stored in a humidor in the dark for 20 minutes, rinsed with 10 mM PBS, then water.
4. Photopolymerization: 650 μL 1:1 polyethylene glycol diacrylate:water, 200 μL 1:1 triethanolamine:water, 50 μL 1-vinyl-2-pyrrolidinone and 100 μL of 3 μM eosin (in 1% methanol) were combined. 60 μL of mixture was added to the array using a rubber well affixed to the surface to contain the liquid. The mixture was irradiated with a 532 nm laser (30-70 mW/cm²) directed from beneath the slide at an angle of ˜75 degrees with respect to surface normal, moving the laser in a circular pattern for 45 seconds. The excess monomer was rinsed off with water and the remaining water was wicked up using a kimwipe.

### Example 2

### Signal Amplification on a DNA Microarray—Mono Hybridization Conditions

A simple DNA microarray was used to evaluate the utility of present invention for signal amplification from a captured oligonucleotide and to quantitatively determine the amplification factor relative to the commonly used horse radish peroxidase (HRP)-conjugated streptavidin used in conjunction with amplex red (Molecular Probes). A simple one-step hybridization, in which an immobilized capture sequence is hybridized to a labeled oligo, was used in this study. The two target oligos were labeled with (a) biotin, and (b) eosin isothiocyanate (EITC).

A sequence designed to be complementary to a highly conserved sequence in the influenza A matrix protein gene served as the negative control capture sequence (amino-C6 terminated 5′, 25 nt spacer, 23 nt sequence). The positive control capture sequence was a randomer (amino-C6 terminated 5′, 25 nt spacer, and 29 nt sequence). For the HRP system, the target oligo was labeled with biotin (Sigma Genosys). For the present system, the target oligo was labeled with EITC (synthesized at Biosource Intl. at the request of the inventors. As the performance of this reaction is obvious to one skilled in the art, it will not be further discussed.) Since all label molecules are of approximately the same size, it was assumed that the hybridization efficiency was the same within error. Each system was hybridized under the same conditions on slides that had been spotted simultaneously. For the present system, monomer was added after hybridization and washing. The polymer was subsequently formed by irradiation. After washing, the polymer was stained with 3 μM eosin in water for 5 minutes, rinsed and imaged. For the HRP system, the biotin-labeled oligos were further labeled with HRP-conjugated strepavidin (Zymed) and diluted 1:1000 in PBS/Tween for a final concentration of 1.25 μg/mL. The substrate was Amplex Red (Molecular Probes) diluted to 50 μM in a PBS, 0.03% hydrogen peroxide solution. A volume of 100 μL was added to the array, and the reaction was allowed to proceed for ˜50 minutes prior to fluorescence quantification.

The effective signal amplification factor was calculated as a ratio of the final signal (e.g., after reaction) to the initial background on that slide after hybridization, assuming that the background signal was representative of no label. Based on this approach, the amplification factor for the HRP system was found to be ˜(2.8±0.2)×10⁵, with error represented as one standard deviation. This value is consistent with widely reported enzymatic amplification factors in the range of 10⁴-10⁶ and confirms that the calculation is reliable. Using the same approach for the present system, the amplification factor was calculated to be (1.06±0.02)×10⁵. Thus, the present invention is competitive in terms of overall amplification.

These steps were followed to obtain the above results:
1. Standard protocols were used to print the DNA capture array.
2. A complimentary oligo labeled with eosin at the 5′ end was synthesized by Biosource Intl. at the request of the inventors and a complimentary oligo labeled with biotin at the 5′ end was purchased from Sigma-Genosys. Both oligos were diluted to 100 μM in Tris buffer.
3. The oligos were diluted to 2.5 μM in Full Moon Hybridization buffer and 10 μL was added to the capture arrays for 2 hours under a coverslip in a humidor. The slides were then washed in 4×SSPE for 5 minutes then water for 5 minutes.
4. Biotin-labeled slides: After applying a rubber well to the slide, 100 μL of Zymed Streptavidin HRP (1.25 μg/mL in 10 mM PBS/0.1% Tween) was added to the well and the slide was placed in a humidor for 1 hour. The slide was washed in 10 mM PBS/0.1% Tween, 10 mM PBS, then water. 100 μL of an amplex red solution (50 μM amplex red and 0.03% hydrogen peroxide in 10 mM PBS) was added to the well and the slide was scanned immediately and at several intervals up to a total reaction time of 50 minutes.
5. Eosin-labeled slides: After applying a rubber well to the slide, 60 μl of monomer mix (325 μL 1:1 polyethylene glycol diacrylate:water, 100 uL 1:1 triethanolamine:water, 25 μL 1-vinyl-2-pyrrolidinone and 50 μL of 3 μM eosin (in 1% methanol)) was added. The mixture was irradiated with a 532 nm laser (30-70 mW/cm²) from beneath the slide at an angle of ˜75 degrees with respect to surface normal, moving the laser in a circular pattern for 45 seconds. The excess monomer was rinsed off with water and the remaining water was wicked up using a kimwipe. The resulting polymer was stained for 5 minutes in 3 μM Eosin in methanol, rinsed with 10 mM PBS then water, and scanned.

### Example 3

### Signal Amplification on an Influenza Microarray

Photopolymerization was achieved for a two-step hybridization (i.e., immobilized capture oligo, target oligo, label oligo with EITC) in which capture and label sequences were designed to target influenza A viruses. In this example, the target, which originated from a patient sample, was bound to the photoinitiating label in solution, and this complex was then hybridized to probes on an influenza microarray. Specifically, the simple influenza microarray contained a positive control capture/label pair to monitor the efficiency of hybridization, a capture/label pair to type influenza A viruses, and a capture/label pair specific for avian A/H5N1 viruses. FIG. 6 contains the fluorescence images (top three images, items 601-609) and transmission images (bottom three images, items 610-618) for an influenza B virus (items 601-603 and 610-612) that served as a negative control, a human influenza A/H1N1 virus (items 604-606 and 613-615), and an avian A/H5N1 virus (items 607-609 and 616-618). The fluorescence images were acquired on the Cy3 channel of a fluorescence-based microarray scanner made by Genetix (retails for ˜$50,000). The transmission images were acquired on a Digital Blue QX-5 microscope, which is a plastic CMOS based microscope that retails for ˜$75. The transmission images are the result of photopolymerization and subsequent staining after hybridization. Prior to photopolymerization and staining no signal can be observed via transmission (as a control).

The positive control indicated good hybridization for each sample—as visualized by items 601, 604, 607, 610, 613, and 616. As expected, the influenza B virus resulted in a negative signal as measured by both fluorescence and transmission—visualized by no signal on items 602, 611, 603 and 612. The A/H1N1 virus, as expected, yielded a positive by both detection methods—as visualized in items 605 and 614. The avian A/H5N1 virus, as expected, yielded a positive signal for both influenza A and H5N1 by both detection methods—as visualized by items 608, 609, 617 and 618. No false positives were observed.

The significance of these results is that influenza virus typing and subtyping using a microarray and standard assay (as in Dawson et al. “MChip: A Tool for Influenza Surveillance” Analytical Chemistry 2006, 78(22), 7610-7615) with the present invention enables the use of an inexpensive reader and practical experimental conditions (in air with visible light excitation), which could lead to improved global influenza surveillance.

These steps were followed to obtain the above results:
1. Virus was extracted from original samples, grown in culture, and the matrix (M) gene segment specifically amplified via PCR by the Centers for Disease Control and Prevention (CDC, Atlanta, Ga.) prior to our receiving them.
2. PCR product of the full length M gene segment from the following 3 samples was then utilized as template in an additional PCR amplification reaction: A/Vietnan/JP36-2/05 (H5N1), A/Bangkok/1544/2004 (H1N1), and B/Mexico/19/2005 (influenza B).
3. PCR reactions were conducted in an Applied Biosystems 9800 Fast thermal cycler utilizing the one-step RT-PCR kit from Qiagen (Valencia, Calif.) under the following conditions: 4 μL full length M gene template, 0.8 μL enzyme mix, 1× final concentration PCR buffer, 0.4 mM final concentration in each dNTP, and 0.6 μM in each primer in a final reaction volume of 20 μL. The reverse PCR primer contained a T7 promoter sequence for subsequent runoff in vitro transcription with T7 RNA polymerase (Invitrogen Corp., Carlsbad, Calif.). PCR cycling conditions were as follows: initial 95° C. for 15 min (to inactivate reverse transcriptase as DNA is used as template as well as to activate the hot-start Taq polymerase), followed by 40 cycles of 94° C. (30 s), 52° C. (30 s), 72° C. (1 min), and a final extension at 72° C. for 10 min.
4. In vitro transcription was subsequently performed using 6 μL PCR product as template, 1 U/μL T7 RNA polymerase, 0.6 U/μL RNase-OUT (Invitrogen Corp., Carlsbad, Calif.), and final concentrations of 1× reaction buffer, 5 μM DTT, and 0.4 mM in each NTP in a total reaction volume of 60 μL
5. The transcribed RNA was fragmented by adding 15 μL of fragmentation buffer (200 mM TRIS-acetate, 500 mM potassium acetate, 150 mM magnesium acetate, pH 8.4) and heating for 25 minutes at 75° C. After flash cooling, the fragmentation was quenched by bringing the solution to 60 mM in EDTA.
6. Standard protocols were used to print the DNA capture array with sequences indicative of influenza A, avian flu H5N1 and a positive control.
7. Labels: Oligos complimentary to the targets or the positive control labeled with eosin at the 3′ end were purchased from Trilink. These oligos were diluted to 100 μM in Tris buffer.
8. 20 μL of the hybridization solution (10 μL fragmented RNA and 10 μL hybridization buffer containing 400 nM target labels and 10 nM control label) was captured for 1 hour under a coverslip in a humidor. The slides were then washed in 2×SSC for 5 minutes then 0.2×SSC for 5 minutes.
9. After scanning with a Genetix scanner, a rubber well was applied to the slide and 60 μL of monomer mix (32% polyethylene glycol diacrylate 575, 11% triethanolamine, 4% 1-vinyl-2-pyrrolidinone, 9% 3 uM eosin (in 1% methanol), and 44% Water) was added. The mixture was irradiated with a 532 nm 40-die hex LED (Norlux) directed from beneath the slide at a distance of about 2 mm for 15 seconds at 15 Hz.
10. The excess monomer was rinsed off with water and the remaining water was wicked up using a kimwipe. The polymer was stained with Congo Red for 5 minutes.

### Example 4

### Response to Number of Photoinitiators Per Unit Area

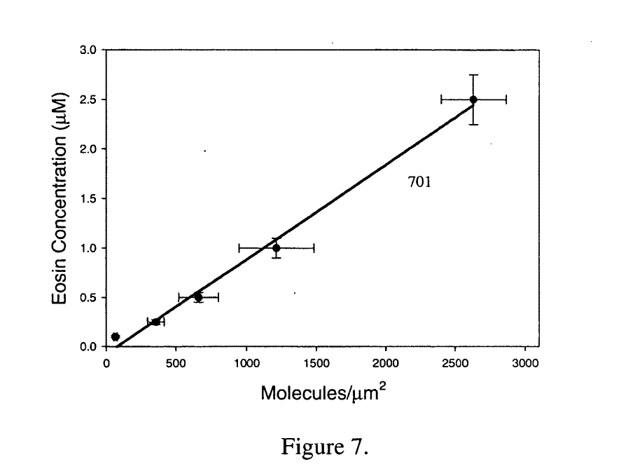

In order to determine the minimum number of EITC molecules needed to achieve significant amplification in the present invention, a series of studies were conducted with amine-terminated oligos and a Cy3 label as an internal standard. The same oligo with an EITC label was spotted simultaneously at exactly the same concentration. The assumption was that the number of molecules remaining on the surface after washing was the same for both systems. Furthermore, Full Moon Cy3 microarray calibration slide was used in conjunction with the Cy3 labeled oligo to calibrate the number of molecules/μm² on the surface. Using the Cy3-labeled oligo as an internal standard, the number of eosin molecules per μm² was determined over a range of spotting concentrations, 0.1-5 μM. The resulting calibration curve of eosin-labeled, amine-terminated oligo spotted onto aldehyde glass is shown in FIG. 7. Error in the y-axis is ±10% based on the error in the stock solution. Error in the x-axis is ±1 standard deviation from 16 measurements. The solid line 701 is a linear regression (R²=0.99) with [E]=(0.07±0.06)+((9.6±0.5)×10-4) (molec/μm²).

From this study, it was determined that the minimum number of photoinitiators required for production of a polymer under the described conditions with visible thickness (i.e., >˜10 μm) was 102±53 per μm² (for the current reagent formulation). This low number of label molecules required on the surface in order to develop an easily visualized signal enables the use of an inexpensive reader and practical experimental conditions (in air with visible light excitation).

These steps were followed to obtain the above results:
1. A 5′ amine-terminated capture oligo that was labeled with eosin at the 3′ end was custom ordered from Biosource Intl. A 5′ amine-terminated capture oligo that was labeled with CY3 at the 3′ end was purchased from Sigma-Genosys. The oligos was diluted to 100 μM in Tris buffer.
2. A range of oligo concentrations were prepared (0.1-5 μM) in 3× Biorad Spotting Buffer. Oligos were spotted at 70% humidity, allowed to react for 24 hours, then washed with 4×SSPE/0.1% SDS, 4×SSPE, water, then 90 degree water. Slides were dried and scanned.

Without wishing to be bound by any specific theory, one possible-photoinitiation mechanism that accounts for the insensitivity to oxygen in the present invention is described herein. As previously detailed, the reagent solution contains two monomers, the monofunctional monomer (M₁) 1-vinyl-2-pyrrolidinone (VP) and a long-chain, difunctional monomer (M₂) poly(ethyleneglycol) diacrylate (PEGDA) having an average length of 575 polyethylene glycol units. The PEGDA provides for structural integrity by means of cross linking, as well as rapid growth of polymer mass from high molecular weight monomers. The VP provides for an enhanced rate of polymer growth because of its lower molecular weight and therefore faster diffusion.

Polymerization is initiated by absorption of light by the photoinitiator eosin (E) in the presence of the co-initiator triethanolamine (TEA) (Cruise et al., 1998; Kizilel et al., 2004). The generally accepted mechanism for photoinitiation is the formation of radicals from TEA by triplet-excited state eosin (³E*):

E+hv→¹E*→³E*. (1)

³E*+TEA→E⁻.+TEA⁺. (2)

Absorption of 532 nm laser light by eosin results in an excited singlet state, which rapidly decays by a combination of fluorescence, internal conversion and intersystem crossing to the triplet state (reaction 1). The quantum yield for triplet formation is high for eosin due to the presence of heavy bromine atoms (http://probes.invitrogen.com/). Triplet-excited eosin will oxidize amine compounds such as TEA and other compounds such as thiols that have low ionization potentials. The radical cation TEA⁺. can deprotonate to from an alkoxy radical (RO.), designated here as TEA. The TEA. radicals thus formed initiate a “living chain polymerization” by adding to the double bonds of the monomers:

TEA.+M→TEA−M. (3)

TEA−M.+M→TEA−M−M. (4)

where M can be M₁ or M₂. Of great importance to the success of photopolymerization is the fate of the eosin radical anion formed in reaction 2. It may undergo further reactions that result in its destruction, in which case the degree of potential polymer growth is greatly reduced, or, in the presence of dissolved oxygen, it may donate an electron to O₂ to form the superoxide anion thus regenerating eosin.

E⁻.+O₂→E+O₂⁻. (5)

The eosin is then available to be photoexcited again, making the initiation of polymerization photocatalytic. The subsequent chemistry of the superoxide radical is quite complex and leads to the formation of H₂O₂, thereby sequestering dissolved oxygen, and to the formation of the highly reactive hydroxyl radical, OH, which can initiate or terminate polymerization.

It is well known that photopolymerization in thin films is generally not possible in ambient air due to termination reactions involving molecular oxygen. For example, free radicals of the monomer (M.) combine with the diradical O₂ and disproportionate to form peroxides:

M.+O₂→MO₂. (6)

MO₂.+MO₂.→MOOM+O₂ (7)

Photopolymerization in bulk solution generally exhibits an induction period during which no photopolymerization occurs (Gou et al., 2004). During this period, dissolved oxygen is consumed in reactions such as reaction 6 and 7. Once the oxygen is removed, photopolymerization begins. In thin films, however, the continuous diffusion of oxygen into the solution prevents polymerization, thereby necessitating the removal of oxygen by purging of the reagent solution and carrying out the reaction in an inert atmosphere.

A remarkable and not yet fully explained property of the eosin/TEA photoinitiation system is that it proceeds in thin films exposed to air (Decker et al., 1979). However, the present invention found that eosin must be present not only as a label on the surface but also at trace levels in solution (typically 0.3 μM) in order to obtain polymer growth at a surface. The requirement of having eosin in the solution itself suggests that it plays a role in removing oxygen. It is hypothesized that the combination of eosin and TEA removes oxygen by means of singlet oxygen (¹O₂*) formation and trapping:

³E*+O₂→¹O₂* (8)

¹O₂*+TEA→Product (9)

Eosin is well known as one of the most efficient singlet oxygen sensitizers (http://probes.invitrogen.com/). The triplet state of eosin is rapidly quenched by O₂ to form ¹O₂* via reaction 8. Amines, especially tertiary amines having α-hydrogens such as TEA, react with ¹O₂*, probably via a charge transfer complex, to form hydroperoxides. Thus, TEA is expected to be a good singlet oxygen trap. By having both eosin and TEA in the solution, oxygen diffusing into the thin film of reagent solution can be continuously removed. Further support of the proposed mechanism may be found it the literature. For example, Decker et al. reported elimination of the oxygen quenching effect on photopolymerization by use of a singlet oxygen sensitizer in combination with the singlet oxygen trap 1,3-diphenylisobenzofuran (Decker et al., 1979). In that system the sensitizer was irradiated at long wavelengths prior to photopolymerization at UV wavelengths.

A simple calculation shows that the rate of removal of oxygen by this mechanism can easily balance the rate at which it diffuses into the solution. The flux of oxygen into the solution is given by Fick's Law:

$${F}_{Diffusion} = - D ( \frac{Δ C}{Δ x} ) = \frac{\begin{matrix}( 2 \times {10}^{- 5} {cm}^{2} {molec}^{- 1} {s}^{- 1} ) ( 0.26 \times {10}^{- 3} mol \cdot {L}^{- 1} ) \\ ( 6.02 \times {10}^{23} molec \cdot {mol}^{- 1} )\end{matrix}}{( 1000 {cm}^{3} \cdot {L}^{- 1} ) ( 0.1 cm )} = 3.1 \times {10}^{13} molec \cdot {cm}^{- 2} \cdot {s}^{- 1}$$

where D is the diffusion coefficient, ΔC is the oxygen concentration gradient, and Δx is the film thickness. For the oxygen concentration gradient, we have chosen the oxygen concentration to be its solubility in water at the air/water interface (0.26 mM) and zero at the microarray surface; the actual liquid layer thickness is 1 mm (65 μL in a 9-mm diameter well).

The flux of photons absorbed by eosin in solution is given by the Beer-Lambert Law under optically thin conditions:

F_{Absorbed}=Iₒεlc=(5.5×10¹⁷ photons·cm²s)(112,000·L·mol⁻¹cm⁻¹)(0.1 cm)(0.32×10⁶ mol·L⁻¹)=2.0×10¹⁷ photons·c.

Here, the photon flux is calculated from the measured laser power of 35 mW at 532 nm expanded over the 9-mm diameter of the well. The path length is the solution thickness of 1 mm, and the eosin concentration used in the bulk solution is 0.32 μM. The ratio of absorbed photons to oxygen molecules diffusing into the solution is F_{absorbed}/F_{Diffusion}=6,500, and thus the combined quantum yield for singlet oxygen formation by eosin and trapping by TEA need only be 1.6×10⁻⁴ in order to maintain the oxygen concentration near zero at the microarray surface. The quantum yield for triplet formation in eosin is about 0.57 (http://probes.invitrogen.com/). Singlet oxygen formation (reaction 8) must compete with reaction of the eosin triplet with TEA (reaction 2) to form radical initiators. The TEA concentration used is 0.78 M compared to the oxygen solubility of 0.26 mM, i.e., about 3000 times greater, but the relative reaction rate with ³E* is unknown. This calculation shows, however, that it is quite feasible that singlet oxygen formation and trapping by TEA may explain the ability of this photopolymerization reaction to proceed in the presence of air, and it suggests possibilities for optimizing the reagent concentrations to achieve improved performance.

The rate law for the hypothesized initiation mechanism outlined in reactions 1-9 is given by:

$$\begin{matrix}Rate = \frac{d [ TEA - M \cdot ]}{d t} \\ = \frac{I {ɛφ}_{T} {k}_{2} {k}_{3} [ E ] [ TEA ] [ M ]}{{k}_{3} [ M ] \{ {k}_{1} [ TEA ] + {k}_{9} [ {O}_{2} ] \}}\end{matrix}$$

where the subscripts for rate constants correspond to the reaction number. This rate law was obtained by applying the steady state approximation to both the eosin triplet state and the TEA. radical. As would be expected, the initial rate increases with laser light intensity, I, absorption cross-section of eosin, ε, quantum yield for triplet formation, φ_{T}, and eosin concentration [E]. The eosin concentration represents both the surface and bulk concentration. The actual rate of the overall polymerization reaction is much more complicated, involving a number of termination reactions, including those with oxygen. A complete analytical expression of initiation, propagation and termination is less informative since it involves a prohibitive number of assumptions. The rate of initiation is the rate of free radical formation, which could be further optimized.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The invention illustratively described herein suitably may be practiced in the absence of any element or elements, limitation or limitations which is not specifically disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims. Whenever a range is given in the specification, all intermediate ranges and subranges, as well as all individual values included in the ranges given are intended to be included in the disclosure. When a Markush group or other grouping is used herein, all individual members of the group and all combinations and subcombinations possible of the group are intended to be individually included in the disclosure.

In general the terms and phrases used herein have their art-recognized meaning, which can be found by reference to standard texts, journal references and contexts known to those skilled in the art. The above definitions are provided to clarify their specific use in the context of the invention.

All patents and publications mentioned in the specification are indicative of the levels of skill of those skilled in the art to which the invention pertains. All references cited herein are hereby incorporated by reference to the extent that there is no inconsistency with the disclosure of this specification. Some references provided herein are incorporated by reference herein to provide details concerning additional starting materials, additional methods of synthesis, additional methods of analysis and additional uses of the invention.

### CITED LITERATURE

Anthony, R. M., Brown, T. J., French, G. L. (2000) “Rapid Diagnosis of Bacteremia by Universal Amplification of 23S Ribosomal DNA Followed by Hybridization to an Oligonucleotide Array” Journal of Clinical Microbiology 38:781-788.
Blanc, N. (2004) “CCD Versus CMOS Image Sensors” http://www.framos.de/pdf_sheets/Blanc-CSEM.pdf
Brammer, L., Fukuda, K., Klimov, A., Cox, N. (2002) “Chapter 5: Influenza” VPD Surveillance Manual, 3^{rd} edition.
Campa, M. (2004) “Molecular Probe's Amplex Red” Product Review.
Constans, A. (2003) “The State of the Microarray/Microarray Instrumentation” The Scientist 17(3):37-42.
Covalciuc, K. A., Webb, K. H., Carlson, C. A. (1999) “Comparison of Four Clinical Specimen Types for Detection of Influenza A and B Viruses by Optical Immunoassay (FLU OIA Test) and Cell Culture Methods” Journal of Clinical Microbiology 37(12):3971-3974.
Cox, N. (2005) John R. LaMontagne Memorial Symposium on Pandemic Influenza Research, Apr. 4-5, 2005.
Cruise, G. M., Hegre, O. D., Scharp, D. S., Hubbell, J. A. (1998) “A Sensitivity Study of the Key Parameters in the Interfacial Photopolymerization of Poly(ethylene glycol) Diacrylate upon Porcine Islets” Biotechnology and Bioengineering 57(6):655-665.
Dawson, E. D., Reppert, A. L, Rowlen, K. L., Kuck, L. R. (2005) “Spotting Optimization for Short Oligo Microarrays on Aldehyde-Glass” Analytical Biochemistry 341:352-360.
Decker, C., Faure, J., Fizet, M., Rychla, L (1979) “Elimination of Oxygen Inhibition in Photo-Polymerization” Photographic Science and Engineering 23(3): 137-140.
Dimmock, N. J., Easton, A. J., Leppard, K. N. (2001) “Introduction to Modern Virology” 5^{th} edition, Blackwell Science Ltd., London.
Doller, G., Schuy, W., Tjhen, K. Y., Stekeler, B. Gerth, H. J. (1992) “Direct Detection of Influenza-Virus Antigen in Nasopharyngeal Specimens by Direct Enzyme-Immunoassay in Comparison with Quantitating Virus Shedding” Journal of Clinical Microbiology 30(4):866-869.
Eisen, M. B., Sabesan, S., Skehel, J. J., & Wiley, D. C. (1997) “Binding of the Influenza A Virus to Cell-Surface Receptors: Structures of Five Hemagglutinin-Sialyloligosaccharide Complexes Determined by X-Ray Crystallography” Virology 26:19-31.
Gold, L., Polisky, B., Uhlenbeck, O., Yarus, M. (1995) “Diversity of Oligonucleotide Functions” Ann. Rev. Biochem. 64:763-797.
Gou, L., Coretsopoulos, C. N., Scranton, A. B. (2004) “Reduction of Oxygen Inhibition in Free-Radical Photopolymerization” Polymer Preprints 45(2):39-40.
Graham, K. C. & Litchfield, D. W. (2000) “The Regulatory Beta Subunit of Protein Kinase CK2 Mediates Formation of Tetrameric CK2 Complexes” Journal of Biological Chemistry 275:5003-5010.
Halder, M., Chowdhury, P. K., Gordon, M. S., Petrich, J. W. (2005) “Hypericin and its Perylene Quinine Analogs: Probing Structure, Dynamics, and Interactions with the Environment” Advances in Photochemistry 28.
Harper, S. A., Fukuda, K., Uyeki, T. M., Cox, N. J., Bridges, C. B. (2005) “Prevention and Control of Influenza” MMWR Recommendations and Reports 54:1-40.
Heller, M. J. (2002) “DNA Microarray Technology: Devices, Systems and Applications” Annu. Rev. Biomed. Eng. 4:129-153.
Hesselberth, J. R., Miller, D., Robertus, J., and Ellington, A. D. (2000) “In vitro Selection of RNA Molecules that Inhibit the Activity of Ricin A-Chain” J. Biol. Chem. 275:1462-1468.
Hubbell, J. A.; Pathak, C. P., Sawhney, A., Desai, N., Hossainy, S., Hill-West, J. L. (2002) http://www.pharmcast.com/Patents/Yr2002/October2002/101502/6465001_Polymer101502.htm
Kern, D., Collins, M., Fultz, T., Detmer, J., Hamren, S., Peterkin, J., Sheridan, P., Urdea, M., White, R., Yeghiazarian, T., Todd, J. (1996) “An Enhanced-Sensitivity Branched-DNA Assay for Quantification of Human Immunodeficiency Virus Type 1 RNA in Plasma” Journal of Clinical Microbiology 34:3196-3203.
Kessler, N., Ferraris, O., Palmer, K., Marsh, W., Steel, A. (2004) “Use of the DNA Flow-Thru Chip, a Three-Dimensional Biochip, for Typing and Subtyping of Influenza Viruses” Journal of Clinical Microbiology 42(5):2173-2185.
Kizilel, S., Perez-Luna, V. H., Teymour, F. (2004) “Photopolymerization of Poly(Ethylene Glycol) Diacrylate on Eosin-Functionalized Surfaces” Langmuir 20:8652-8658.
Kothapalli, R., Yoder, S. J., Mane, S., Loughran, T. P. (2002) “Microarray Results: How Accurate are They?” Bioinformatics 3:22.
Lamb, R. A. & Krug, R. M., (1996) “Orthomyxoviridae: The Viruses and their Replication, in Fields Virology”, B. N. Fields, D. M. Knipe, and P. M. Howley, Editors. Lippincott-Raven: Hagerstown.
Li, J., Chen, S., & Evans, D. H. (2001) “Typing and Subtyping Influenza Virus Using DNA Microarrays and Multiplex Reverse Transcriptase PCR” Journal of Clinical Microbiology 39:696-704.
Love, K. R., Andrade, R. B., & Seeberger, P. H. (2001) Linear Synthesis of a Protected H-Type II Pentasaccharide Using Glycosyl Phosphate Building Blocks. Journal of Organic Chemistry 66:8165-8176.
Marcaurell, L. A. & Seeberger, P. H. (2002) “Combinatorial Carbohydrate Chemistry” Current Opinions in Chemical Biology 6:289-296.
McInnes, J. L., Forster, A. C., Skingle, D. C., Symons, R. H. (1990) “Preparation and uses of photobiotin” Methods Enzymol. 184:588-600.
Mehlmann, M., Townsend, M., Stears, R., Kuchta, R., Rowlen, K. L. “Optimization of Fragmentation Conditions for Microarray Analysis of Viral RNA” (2005) submitted to Analytical Biochemistry.
Mir-Shekari, S. Y., Ashford, D. A., Harvey, D. J., Dwek, R. A., & Schulze, I. T. (1997) “The Glcosylation of the Influenza A Virus Hemagglutinin by Mammalian Cells” Journal of Biological Chemistry 272:4027-4036.
Na, S. Y., Choi, J. E., Kim, H. J., Jhun, B. H., Lee, Y. C., & Lee, Y. W. (1999) “Bcl3, an IkappaB Protein, Stimulates Activating Protein-1 Transactivation and Cellular Proliferation” Journal of Biological Chemistry 274:28491-28496.
Nadon, R., Shoemaker, J., (2002) “Statistical Issues with Microarrays: Processing and Analysis” Trends Genet. 18:265-271.
Nallur, G., Luo, C., Fang, L., Cooley, S., Dave, V., Lambert, J., Kukanskis, K., Kingsmore, S., Lasken, R., Schweitzer, B. (2001) “Signal Amplification by Rolling Circle Amplification on DNA Microarrays” Nucleic Acids Research 29:E118.
Nimjee, S. M., Rusconi, C. P., & Sullenger, B. A. (2005) “APTAMERS: An Emerging Class of Therapeutics” Ann. Rev. Med. 56:555-583.
Palese, P., Basler, C. F., Garcia-Sastre, A. (2002) “The Makings of a Killer” Nature Medicine 8(9):927-928.
Robertson, S. A., Harada, K., Frankel, A. D., and Wemmer, D. E. (2000) “Structure Determination and Binding Kinetics of a DNA Aptamer-Argininamide Complex” Biochemistry 39:946-954.
Schena, M. (2003) “Microarray Analysis” Wiley-Liss, Hoboken, N.J.
Sengupta, S., Onodera, K., Lai, A., Melcher, U. (2003) “Molecular Detection and Identification of Influenza Viruses by Oligonucleotide Microarray Hybridization” Journal of Clinical Microbiology 41(10):4542-4550.
Smagala, J. A., Dawson, E. D., Mehlmann, M., Townsend, M. B., Kuchta, R. D., Rowlen, K. L. (2005) “ConFind: a Robust Tool for Conserved Sequence Identification” submitted to Bioinformatics.
Smith, C. (2005), CDC, Influenza Branch, personal communication.
Stears, R. L., Getts, R. C., Gullans, S. R. (2000) “A Novel, Sensitive Detection System for High-Density Microarrays using Dendrimer Technology” Physiol Genomics 3:93-99.
Steet, R. A., Melançon, P., & Kuchta, R. D. (2000) “3′-Azidothymidine Potently Inhibits the Biosynthesis of Highly Branched N-linked Oligosaccharides and Poly-N-acetyllactosamine Chains in Cells” Journal of Biological Chemistry 275:26812-26820.
Taussig, M. J., Landegren, U. (2003) “Progress in Antibody Arrays” Targets 2(4):169-176.
Tsuchida, A., Kobayashi, K., Matsubara, N., Muramatsu, T., Suzuki, T., & Suzuki, Y. (1998) “Simple Synthesis of Sialyllactose-carrying Polystyrene and Its Binding with Influenza Virus” Glycoconjugate Journal 15:1047-1054.
Tuerk, C., MacDougal, S., Gold, L. (1992) “RNA Psuedoknots that Inhibit HIV Type I Reverse Transcriptase” Proc. Natl. Acad. Sci. 89:6988-6972.
Vernet, G. (2002) “DNA-Chip Technology and Infectious Diseases” Virus Research 82:65-71.
Wang, D., Coscoy, L., Zylberberg, M., Avila, P. C., Boushey, H. A., Ganem, D., DeRisi, J. L. (2002) “Microarray-Based Detection and Genotyping of Viral Pathogens” Proceedings of the National Academy of Sciences of the United States of America 99(24):15687-15692.
Wang, X., Ghosh, S., Guo, S. (2001) “Quantitative Quality Control in Microarray Image Processing and Data Acquisition” Nucleic Acids Research 29(15):41.
Ward E., Foster, S. J., Fraaije, B. A., McCartney, H. A. (2004) “Plant Pathogen Diagnostics:Immunological and Nucleic Acid-Based Approaches” Ann. Appl. Biol. 145: 1-16.
Webster, R. G., Bean, W. J., Gorman, O. T., Chambers, T. M., & Kawaoka, Y. (1992) Evolution and Ecology of Influenza A Viruses. Microbiology Reviews 56:152-179.
Webster. R. G., Cox, N. J., Stohr, K. (2002) “WHO Manual on Animal Influenza Diagnosis and Surveillance” WHO/CDS/SCR/NCS/2002.5 Rev. 1.
Weiss, S. (2002) “Choosing Components for a Microarray Scanner”
http://sales.hamamatsu.com/assets/applications/Combined/genescanner.pdf
Xu, X., Smith, C. B., Mungall, B. A., Lindstrom, S. E., Hall, H. E., Subbarao, K., Cox, N. J., Klimov, A. (2002) “Intercontinental Circulation of Human Influenza A(H1N2) Reassortant Viruses During the 2001-2002 Influenza Season” Journal of Infectious Diseases 186(10):1490-1493.
Zou, S. (1997) “A Practical Approach to Genetic Screening for Influenza Virus Variants” Journal of Clinical Microbiology 35(10):2623-2627.

1-37. (canceled)

38. A kit for identifying a target by amplifying a molecular or biomolecular recognition interaction between a target and a probe comprising:
a polymerizing solution comprised of a polymer precursor, wherein the polymerizing solution allows polymer growth in a region surrounding a photoinitiator label without an oxygen-reducing purge.

39. The kit of claim 38, wherein the polymerizing solution includes an amine co-initiator and a photoinitiator, wherein the photoinitiator is provided in a sufficient amount in the polymerizing solution to allow the polymer growth in the region surrounding the photoinitiator label without an oxygen-reducing purge.

40. The kit of claim 39, wherein the photoinitiator is provided in the sufficient amount in the polymerizing solution to allow polymer growth in the region surrounding the photoinitiator label without an oxygen-reducing purge of any of the polymerizing solution, components of the polymerizing solution, and atmosphere surrounding the polymerizing solution.

41. The kit of claim 39, wherein the polymer precursor is PEGDA, the amine co-initiator is TEA, and the photoinitiator contains eosin.

42. The kit of claim 40, further comprising a photoinitiator label containing eosin.

43. The kit of claim 41, further comprising a photoinitiator label containing eosin.

44. The kit of claim 38, wherein a photoinitiator label further includes at least one of avidin and streptavidin, and wherein the target is labeled with biotin, so as to allow interaction between the biotin and the at least one of avidin and streptavidin to attach the photoinitiator label and the target labeled with biotin to one another.

45. The kit of claim 38, wherein a photoinitiator label further includes a plurality of photoinitiators.

46. The kit of claim 38, wherein the polymerizing solution is configured for irradiation with light in the visible spectrum to initiate formation of a solid polymer.

47. The kit of claim 46, wherein the visible light has a wavelength of about 532 nm.

48. The kit of claim 38, wherein the probe is attached to a solid substrate.

49. The kit of claim 48, wherein the solid substrate includes a plurality of probes forming a microarray.

50. The kit of claim 38, further comprising at least one of a) the polymer formed is a fluorescent polymer, a chromophoric polymer, a chemiluminescent polymer, a light scattering polymer, a stained polymer, a magnetic polymer, a radioactive polymer, and an electrically conducting polymer, and b) the polymer formed is contacted with a solution comprising at least one of a fluorophore, a chromophore, a chemiluminophore, a light scattering material, a magnetic material, a radioactive material, and an electrically conductive material, thereby allowing absorption of the solution by the polymer, and removing excess of the at least one of the fluorophore, the chromophore, the chemiluminophore, the light scattering material, the magnetic material, the radioactive material, and the electrically conductive material of the solution from the polymer.

51. A method of identifying a target by amplifying a molecular or biomolecular recognition interaction between a target and a probe comprising the steps of:
a) forming a photoinitiator label-target-probe complex;
b) substantially removing any unbound photoinitiator label;
c) contacting the photoinitiator label-target-probe complex with a polymerizing solution comprised of a polymer precursor, wherein the polymerizing solution allows polymer growth without an oxygen-reducing purge;
d) exposing the photoinitiator label-target-probe complex and the polymerizing solution to light, so as to form a polymer in a region surrounding the photoinitiator label, without an oxygen-reducing purge.
e) detecting the polymer formed, thereby detecting an amplified target-probe interaction.

52. The method of claim 51, wherein contacting the photoinitiator label-target-probe complex with the polymerizing solution includes providing the polymerizing solution comprised of the polymer precursor, an amine co-initiator, and a photoinitiator, wherein the photoinitiator is provided in a sufficient amount in the polymerizing solution to allow the polymer growth in the region surrounding the photoinitiator label without an oxygen-reducing purge.

53. The method of claim 52, wherein contacting the photoinitiator label-target-probe complex with the polymerizing solution includes providing the polymerizing solution comprised of the polymer precursor, the amine co-initiator, and the photoinitiator, wherein the photoinitiator is provided in a sufficient amount in the polymerizing solution to allow the polymer growth in the region surrounding the photoinitiator label without an oxygen-reducing purge of any of the polymerizing solution, components of the polymerizing solution, and atmosphere surrounding the polymerizing solution.

54. The method of claim 51, wherein exposing the photoinitiator label-target-probe complex and the polymerizing solution to the light in the visible spectrum, so as to form the polymer in the region surrounding the photoinitiator label, without an oxygen-reducing purge of any of the polymerizing solution, components of the polymerizing solution, and atmosphere surrounding the polymerizing solution.

55. The method of claim 51, wherein the polymer precursor is PEGDA, the amine co-initiator is TEA, and the photoinitiator contains eosin.

56. The method of claim 51, wherein the photoinitiator label contains eosin.

57. The method of claim 51, wherein the target is labeled with biotin, the photoinitiator label comprises at least one of avidin and streptavidin, so as to allow interaction between the biotin and the at least one of avidin and streptavidin to attach the photoinitiator label and the target labeled with biotin to one another.

58. The method of claim 51, wherein the photoinitiator label contains a plurality of initiators.

59. The method of claim 51, wherein the probe is attached to a solid substrate.

60. The method of claim 59, wherein the solid substrate includes a plurality of probes forming a microarray.

61. The method of claim 51, wherein nonspecific interactions between the substrate and either of the target and the photoinitiator label are limited by application of a blocking agent to the substrate during step a) or by use of a crowding agent during step a).

62. The method of claim 51, further comprising the step of removing unpolymerized polymer precursor prior to detecting polymer formation.

63. The method of claim 60, further comprising the step of removing unpolymerized polymer precursor prior to detecting polymer formation.

64. The method of claim 62, further comprising the step of removing unpolymerized polymer precursor prior to detecting polymer formation.

65. The method of claim 51, wherein the intensity of the light used during step d) is fluctuated.

66. The method of claim 51, wherein at least one of a) the polymer formed is a fluorescent polymer, a chromophoric polymer, a chemiluminescent polymer, a light scattering polymer, a stained polymer, a magnetic polymer, a radioactive polymer, and an electrically conducting polymer, and b) the polymer formed is contacted with a solution comprising at least one of a fluorophore, a chromophore, a chemiluminophore, a light scattering material, a magnetic material, a radioactive material, and an electrically conductive material, and removing excess of the at least one of the fluorophore, the chromophore, the chemiluminophore, the light scattering material, the magnetic material, the radioactive material, and the electrically conductive material of the solution from the polymer.
